(19) United States
(12) Plant Patent Application Publication
(10) Pub. No.: US 2020/0288613 P1
(43) Pub. Date: Sep. 10, 2020

(54) Apple Tree Named 'Takano 6'

(50) ;

(71) Applicant: Tsuyoshi TAKANO, Oshu (JP)

(72) Inventor: Tsuyoshi TAKANO, Oshu (JP)

(21) Appl. No.: 16/873,248

(22) Filed: Mar. 4, 2020

Related U.S. Application Data
(60) Provisional application No. 62/815,525, filed on Mar. 8, 2019.

(51) Int. Cl. A01H 6/74

(52) U.S. Cl. PLT/161

(57) ABSTRACT

A new and distinct apple variety is described, the variety resulting from controlled cross pollination between (Fuji x Shinano Gold). This variety can be characterized by its full red coloration and crisp texture. The new variety has been named ‘Takano 6’.

Genus and species plant claimed: Malus domestica.

Variety denomination: ‘Takano 6’.

### BACKGROUND OF THE INVENTION

The new variety of apple tree, Malus domestica, resulted from controlled cross pollination between (Fuji x Shinano Gold). The new variety was determined to be distinct from the parent varieties.

### SUMMARY OF THE INVENTION

This variety can be characterized by its full red coloration and crispy texture. The variety is also different in that the harvest time is one week earlier and higher acidity than Fuji. The new variety has been named ‘Takano 6’. Takano 6 is currently only grown in Japan under Japanese conditions and has been bred by a private breeder.

### BRIEF DESCRIPTION OF THE DRAWINGS

The accompanying photographs show typical specimens of the tree, foliage and fruit of the new variety as depicted in colors as nearly true as is reasonably possible to make the same in a color illustration of this character.

FIG. 1 shows the tree in flower.

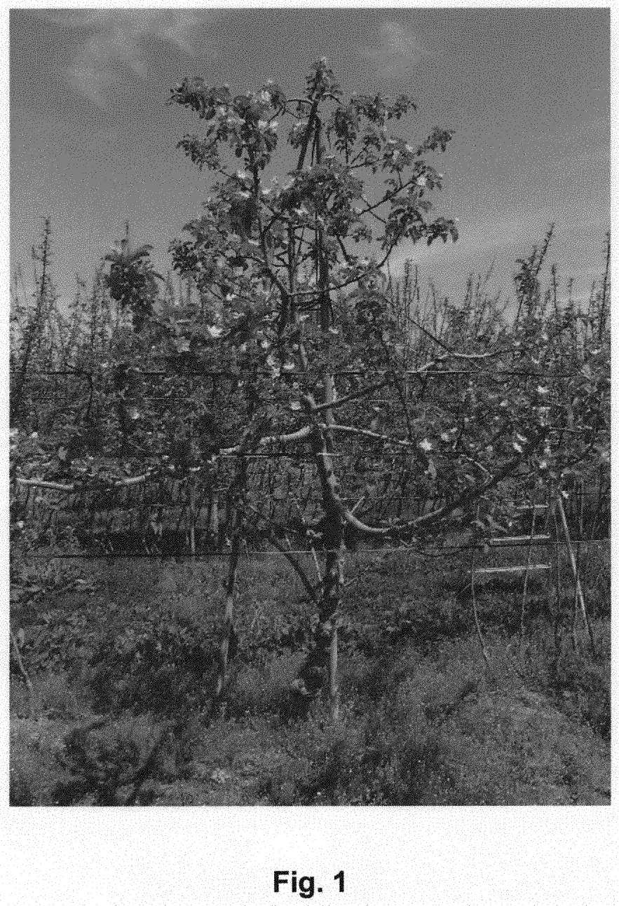

FIG. 2 shows flowers from above, below and in section.

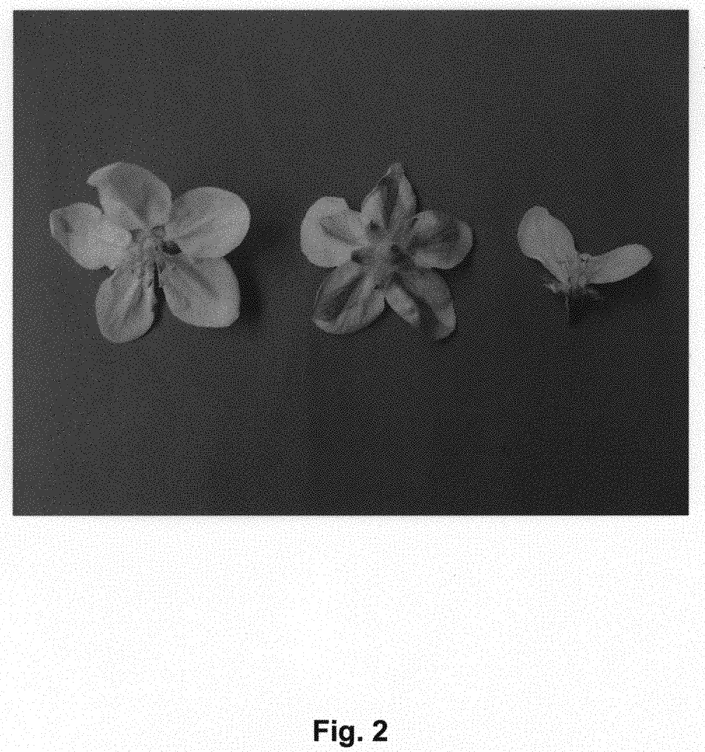

FIG. 3 shows detached flower parts.

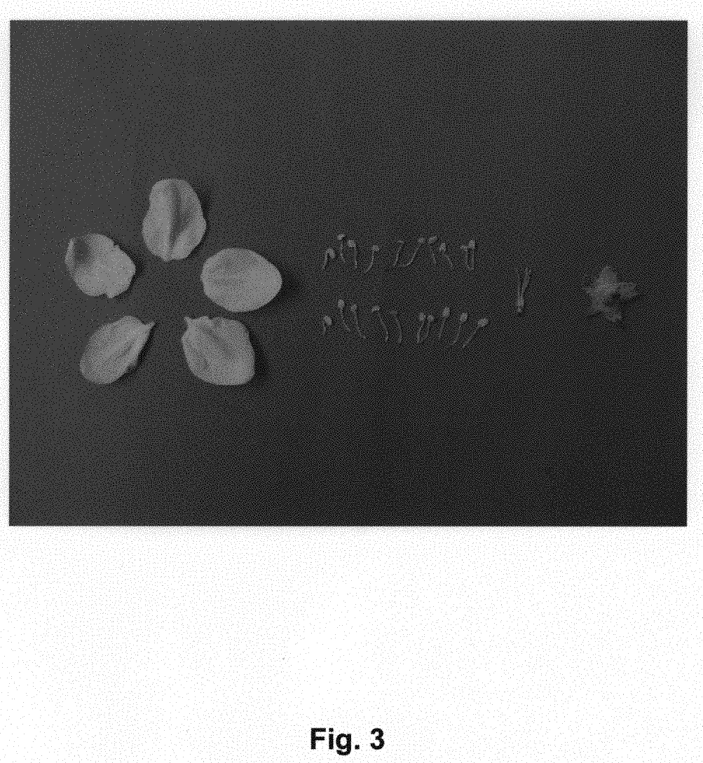

FIG. 4 shows the tree in fruit.

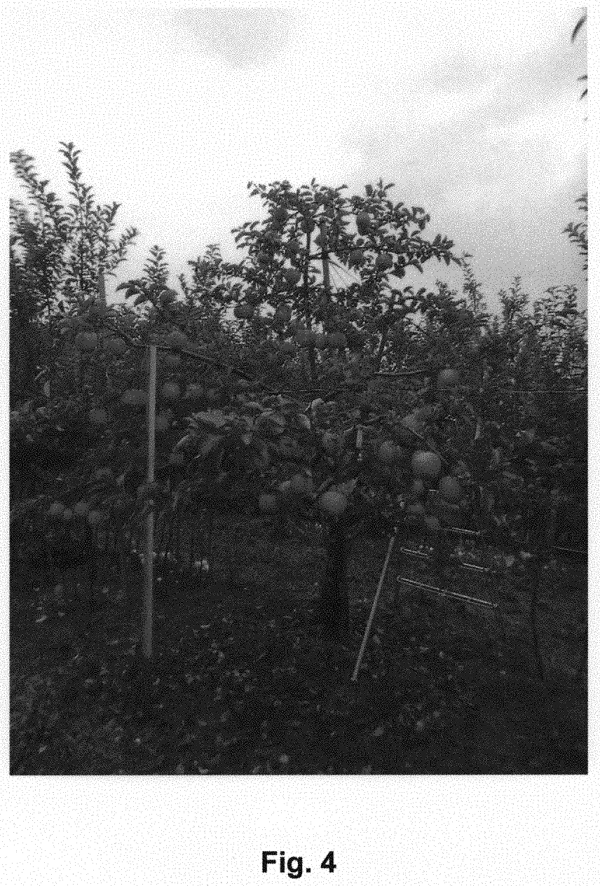

FIG. 5 shows fruit in transverse and vertical section.

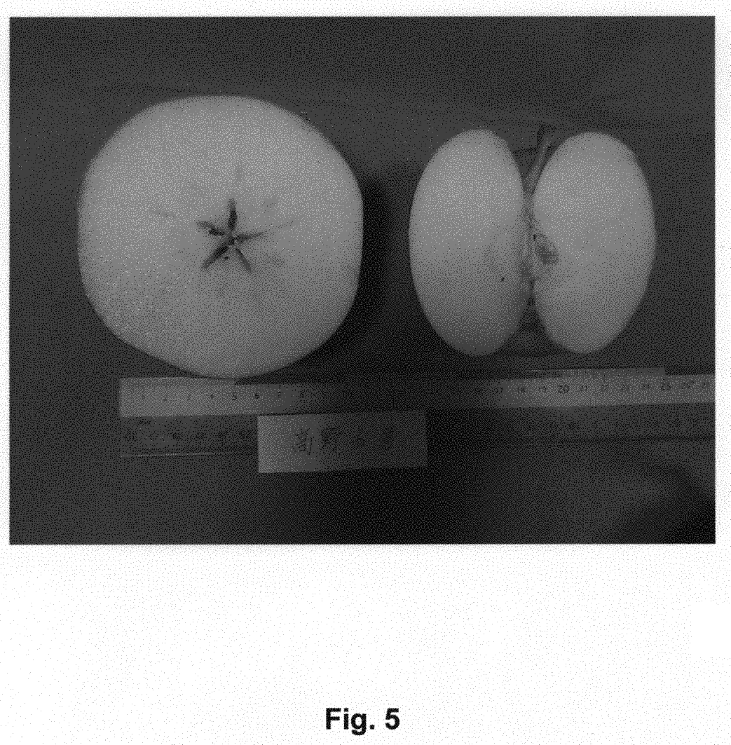

FIG. 6 shows several fruit from above.

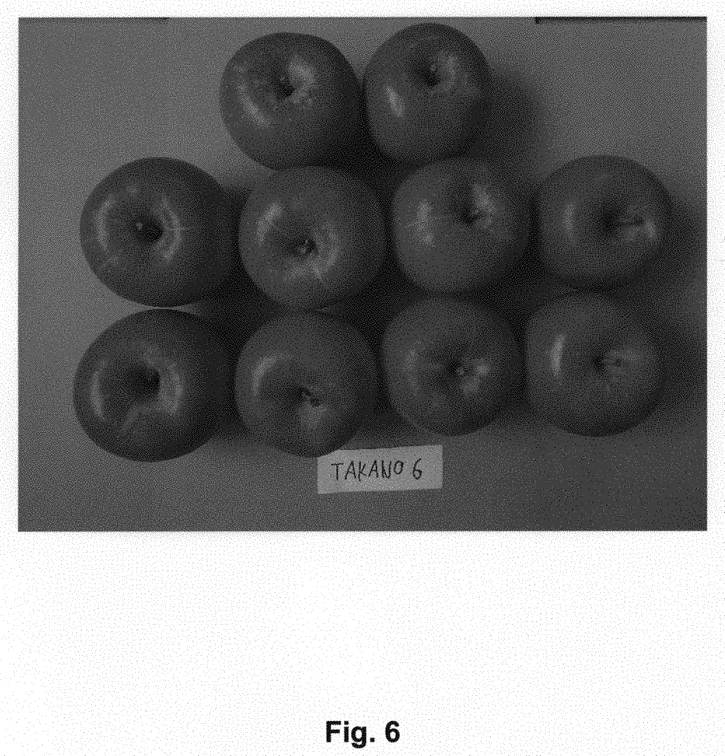

FIG. 7 shows several fruit from a side view.

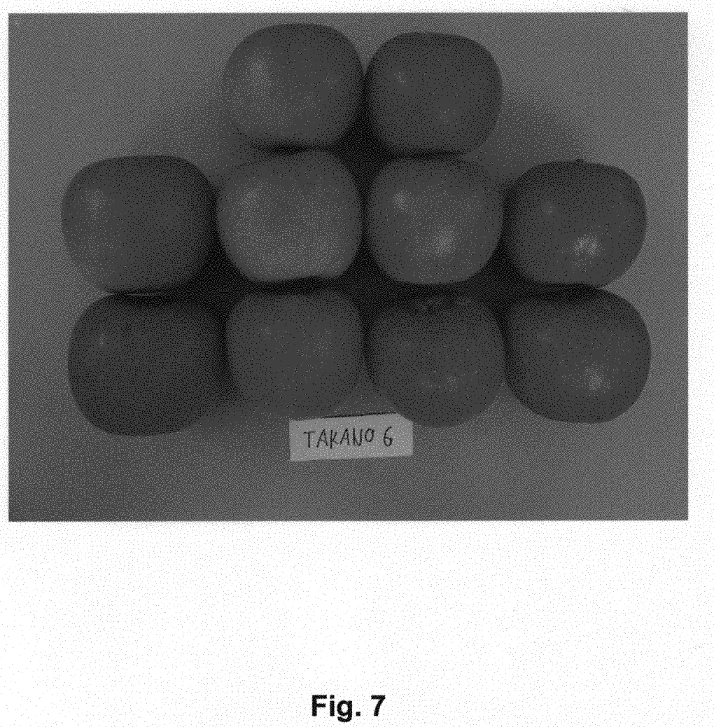

FIG. 8 shows several fruit from above.

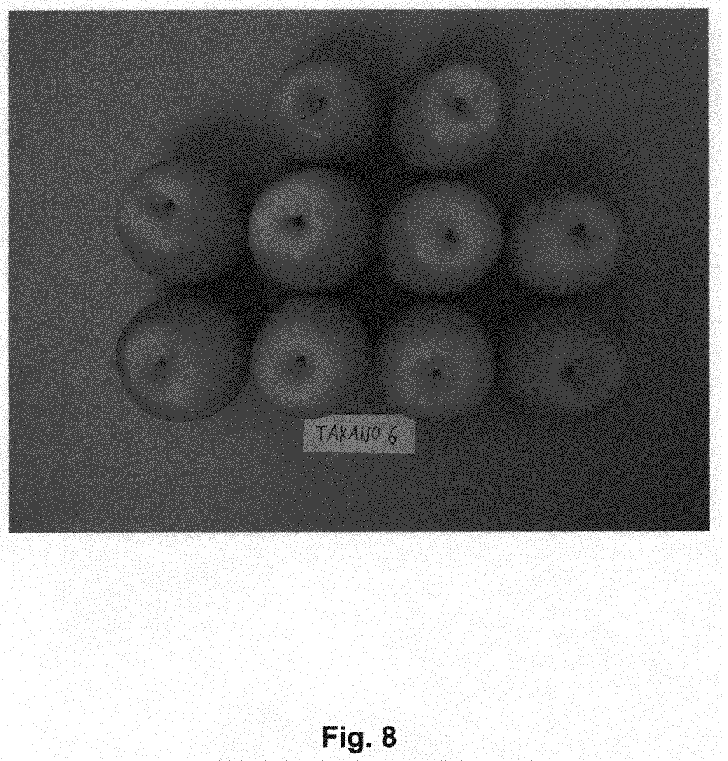

FIG. 9 shows fruit from above and in side view.

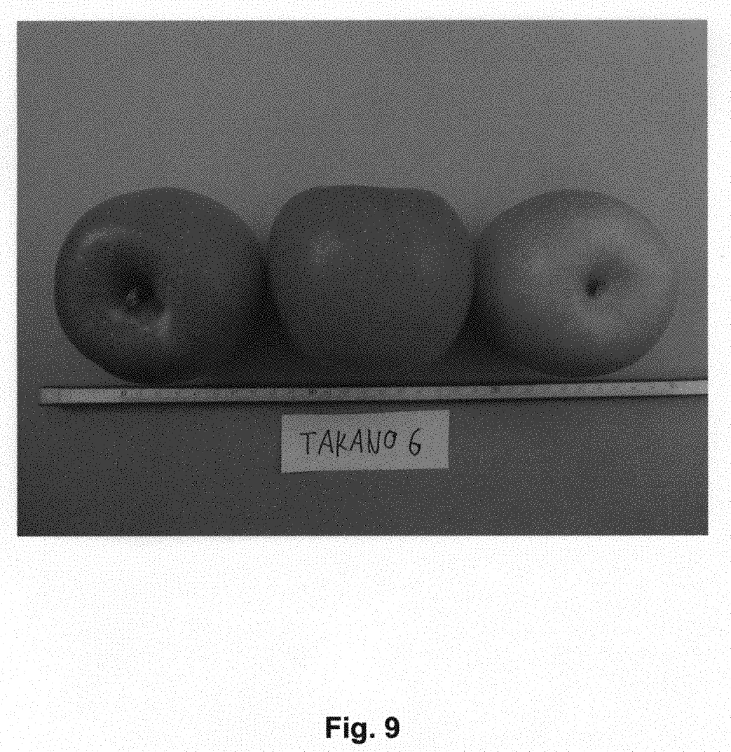

### DETAILED DESCRIPTION

The following is a detailed description of the new variety:

Information on the breeding scheme and propagation of the variety. Breeding: Produced by controlled pollination; Parents: (Fuji x Shinano Gold). Method of propagating the variety through vegetative propagation: Grafting.

Characteristics of the variety. a) Tree Type: ramified. b) Tree Habit: spreading.

c) Fruit general shape: globose˜obloid. d) Relative area of fruit over color: Large (similar to Jonagold).

e) Time of beginning of flowering (10% open flowers): Medium (same as Fuji).

f) Time of eating maturity: late (one week earlier than Fuji in Japan).

g) Other characteristics of the variety: Duration of storage: Variety has long term storage potential in regular air.

Detailed characteristics are shown in the table below:

| Characteristics | State |
|---|---|
| One-year-old shoot thickness | medium (5.1 mm) |
| One-year-old shoot: length of internode | short (3.3 cm) |
| One-year old shoot: color on sunny side | medium brown |
| One-year-old shoot: pubescence (on distal half of shoot | medium |
| One-year-old shoot: number of lenticels | many |
| Leaf blade: attitude in relation to shoot | outwards |
| Leaf blade: length | medium (10.7 cm) |
| Leaf blade: width | medium (6.5 cm) |
| Leaf blade: ratio length/width | medium (1.65) |
| Leaf blade: intensity of green color | medium |
| Petiole: length | medium (2.6 cm) |
| Petiole: extent of anthocyanin coloration from base | Large |
| Flower: predominant color at balloon stage | light pink |
| Flower: diameter with petals pressed into horizontal | medium (4.0 cm) |
| position | |
| Flower: arrangement of petals | Intermediate |
| Flower: position of stigmas relative to anthers | Same level |
| Young fruit: extent of anthocyanin overcolor | medium |
| Fruit: size | large (383 g) |
| Fruit: height | tall (8.5 cm) |
| Fruit: diameter | large (9.6 cm) |
| Fruit: ratio height/diameter | large (0.88) |
| Fruit: ribbing | absent or weak |
| Fruit: crowning at calyx end | absent or weak |
| Fruit: size of eye | small |
| Fruit: bloom of skin | absent or weak |
| Fruit: greasiness of skin | absent of weak |
| Fruit: surface texture of skin | medium |
| Fruit: ground color | yellow |
| Fruit: hue of over color-with bloom removed | red |
| Fruit: intensity of over color | medium- |
| Fruit: pattern of over color | Solid flush with |
| | weakly |
| | defined stripes |
| Fruit: width of stripes | medium |
| Fruit: intensity of over color | weak |
| Fruit: area of russet around stalk attachment | medium |
| Fruit: area of russet on cheeks | absent or weak |
| Fruit: area of russet around eye basin | absent or small |
| Fruit: number of lenticels | medium |
| Fruit: size of lenticels | small |
| Fruit: scarfskin | absent |
| Fruit: length of stalk | medium (2.1 cm) |
| Fruit: thickness of stalk | medium (1.9 mm) |
| Fruit: depth of stalk cavity | deep (1.9 cm) |
| Fruit: width of stalk cavity | medium (4.4 cm) |
| Fruit: depth of eye basin | medium (1.1 cm) |
| Fruit: width of eye basin | medium (3.6 cm) |
| Fruit: firmness of flesh | firm (14.5 lbs) |
| Fruit: color of flesh | cream |
| Fruit: sweetness of flesh | High (16.0%) |
| Fruit: acidity of flesh | Medium (0.41%) |
| Fruit: water-core of flesh | Absent-very slight |
| Fruit: shape of core | conical |
| Fruit: aperture of locules (in transverse section) | moderately open |

I claim:

1. A new and distinct variety of apple tree as illustrated and described herein.
